(19) United States
(12) Patent Application Publication
(10) Pub. No.: US 2003/0054544 A1
(43) Pub. Date: Mar. 20, 2003

(54) Oxygen enriched bioreactor and method of culturing cells

(75) Inventor: Micheal Gruenberg, Poway, CA (US)

Correspondence Address: KINNEY & LANGE, P.A., THE KINNEY & LANGE BUILDING, 312 SOUTH THIRD STREET, MINNEAPOLIS, MN, 55415-1002, US

(73) Assignee: MedCell Biologics, Inc., San Diego, CA

(21) Appl. No.: 09/952,463

(22) Filed: Sep. 14, 2001

(51) Int. Cl. C12M 3/06

(52) U.S. Cl. 435/289.1; 435/297.2; 435/297.4

(57) ABSTRACT

The present invention includes a bioreactor support system that provides an improved way to culture large quantities of mammalian cells. Mammalian cells are extremely sensitive to their environment, undergoing a programmed cell death in response to nutrient deprivation, growth factor withdrawal, oxygen starvation and excess shear levels. The present invention includes a bioreactor support system which permits the culture of cells at high density and prevents cell death by providing enhanced oxygen delivery to the cells without excessive shear; maintaining cell secreted or exogenously added low molecular weight products (e.g., growth, differentiation and maintenance factors) in the culture space during perfusion culture; and provides the ability to feed the cultures without dilution of the conditioned media. The improved bioreactor support system features two flow paths emanating from a central integrating chamber. The first flow path is a slow speed loop from a bioreactor containing a cell retention module and a return line for cells. Cell-free media from the cell retention module enters the integrating chamber. The second flow path is a high speed loop containing an oxygenation module and a dialysis module. The return line from the integrating chamber to the bioreactor is a rate controlled perfusion system containing oxygen-saturated, pH adjusted, nutrient-rich, metabolic waste depleted, conditioned media. The support system can be used to enhance the growth and maintenance of mammalian cells in a variety of bioreactor configurations, including fermentors, spinner flasks, flexible bags, rotational devices and hollow fibers.

### BACKGROUND OF THE INVENTION

The present invention generally relates to a system and method for growing or maintaining biological cells in vitro. More specifically, the present invention relates to a bioreactor system and method that are effective to grow or maintain biological cells at a high cell density, while providing adequate oxygen and nutrients to, and removing waste products, from the biological cells.

Mammalian cells are increasingly used in diagnostic and medical applications. For example, mammalian cells may be used for production of proteins for vaccines, therapeutics and diagnostics. In addition, mammalian cells may be used for adoptive cell therapy or tissue engineering. Furthermore, mammalian cells may be used as part of a medical device that functions as an artificial organ. As a result, there is an increase in demand for mammalian cells and/or mammalian cell-based products. The increase in demand has fueled a need for techniques and/or equipment that promote efficient mammalian cell growth and/or productivity.

Bioreactors have commonly been employed for the cultivation of living organisms, such as mammalian cells. A bioreactor typically includes a housing that contains cells and nutrients maintained at bioreactor conditions that permit cell growth and/or production of secreted products. Furthermore, bioreactors used for mammalian cell cultures are well known in the art, and are commercially available from a variety of manufacturers (see, Maisenholder, G., The Scientist 13(14):21 1999). As an example, bioreactors may include spinner flasks, roller bottles (see U.S. Pat. No. 8,866,419 issued to Meder), hollow fibers (see U.S. Pat. No. 3,997,396 issued to Knazek), stirred tank fermenters, gas permeable bags (see U.S. Pat. No. 6,190,913 issued to Singh) and porous bed reactors (see U.S. Pat. No. 5,510,262 issued to Stephanopoulos).

Cell culture efficiency and productivity of mammalian cells are generally related to (1) supply of oxygen, (2) supply of nutrients and removal of waste products, and (3) control of the cell micro-environment.

Supply of Oxygen

The growth and culture of mammalian cells typically require a constant supply of adequate oxygen. Oxygen diffusion in culture media is a function of a liquid-to-air surface area when operating the bioreactor. Furthermore, oxygen transfer is limited by the liquid-to-air surface area and any shear forces created by agitation and/or sparging. In addition, as the bioreactor volume increases, the ability to adequately supply oxygen is diminished.

As a result, several methods have been employed to provide adequate oxygen to bioreactors to permit scale-up for cell culture production. The methods include agitation (see U.S. Pat. No. 6,199,913 issued to Singh), sparging by introducing gas bubbles into airlift fermenter devices or oxygenating by diffusion through gas permeable tubing (see U.S. Pat. No. 5,112,760 issued to Baumgartner and U.S. Pat. No. 5,081,035 issued to Halberstadt). Oxygen diffusion may also be increased through increased pressurization of the bioreactor headspace.

Furthermore, addition of a draft tube has been used to enhance axial mixing in a bioreactor and reduce bubble coalescence that result in smaller bubbles. Smaller bubbles are desirable in bioreactors since smaller bubbles produce less shear and increase a rate of oxygen transfer and/or oxygen diffusion. Nonetheless, even with these methods, adequate oxygen supply is still a major impediment to scaling-up mammalian cell cultures produced in bioreactors.

When bioreactor volumes are greater than three liters, air sparging is required for effective oxygen transfer since introducing bubbles into a culture media by sparging results in a dramatic increase in the liquid-to-air surface area. In addition, agitation is used to break up the bubbles to thereby further increase oxygen transfer.

Unfortunately, both bubbling and agitation typically have a detrimental effect on biological cells, such as mammalian cell cultures. Biological cells may be rendered non-viable through bubble breakup and/or coalescence within the culture media, especially at a surface gas-to-liquid interface. Therefore, maximizing oxygen transfer in the bioreactor must be balanced by maintaining cell viability.

Supply of Nutrients and Removal of Waste Products

Mammalian cell cultures typically utilize glucose as an energy source. When glucose is limited, glutamine becomes a predominant energy source. As a result, most culture media include glucose as a primary nutrient component along with glutamine and other types of amino acids. Under typical cell culture conditions, mammalian cells metabolize (1) glucose into lactate, and (2) glutamine into ammonia. Unfortunately, accumulation of metabolic waste products, such as lactate and ammonia in cell cultures greatly restricts cell concentration, cell growth and cell viability.

When biological cells are grown in the bioreactor, such as a roller bottle, a flask, a bag or a fermentor, the bioreactor may be operated in a batch culture mode. In the batch culture mode, the bioreactor is inoculated with cells at a starting concentration of between about 0.1 million cells per milliliter (cells per mL) and 0.5 million cells per mL of culture media. Next, the cells are grown to confluence or to about 1 million cells per mL to about 2 million cells per mL. After reaching about 1 million cells per mL to about 2 million cells per mL, cell growth is terminated, and contents of the bioreactor are harvested.

When the number of cells for inoculation is limited, the bioreactor can be operated in a fed-batch culture mode. In the fed-batch culture mode, cells are inoculated at a same concentration as in the batch culture mode, but into a lower volume than a maximum liquid bioreactor volume. Next, culture media is continuously added to the bioreactor until the maximum liquid bioreactor volume is reached. Both batch culture and fed-batch culture modes are the least efficient modes of operating a bioreactor, since both culture modes do not have provision for removal of metabolic waste products like lactate and ammonia.

However, accumulation of metabolic waste products can be reduced by changing from the batch culture or the fed-batch culture modes to a continuous perfusion culture mode. In the continuous perfusion culture mode, fresh culture media is continuously added to the bioreactor, while culture media already present in the bioreactor that contains cells, secreted products, exogenously added factors, metabolic waste products and/or unused nutrients are continuously removed. As a result, the continuous perfusion culture mode enables (1) continuous removal of metabolic waste products, (2) replenishment of nutrients, and (3) an extended life for cell cultures. In addition, bioreactor productivity, as measured in weight per volume per unit of time, is significantly higher in the continuous perfusion culture mode rather than the batch culture or fed-batch culture modes.

However, in order to maintain adequate levels of nutrients and avoid accumulation of metabolic waste products, large volumes of culture media must be perfused through the bioreactor. The large volume requirement of culture media makes the continuous perfusion culture mode both expensive and labor-intensive. In addition, beneficial products, such as the biological cells and exogenously added factors are also removed with the metabolic waste products. Removal of beneficial products from the bioreactor decreases the efficiency of the bioreactor operating in the continuous perfusion culture mode.

As an example, the continuous perfusion culture mode may operate in a chemostat design. A bioreactor operating in a continuous chemostat mode is programmed to maintain a constant volume in the bioreactor by continuously feeding culture media into the bioreactor and continuously removing waste culture media from the bioreactor. Maintaining the constant volume in the bioreactor may be accomplished by using an overflow device that causes excess culture media to overflow when the bioreactor volume rises above the overflow device. In addition, maintaining the constant volume in the bioreactor may also be accomplished with matched pumps for feeding the culture media into the bioreactor and removing the waste media from the bioreactor. Furthermore, the feed rate for the culture media can be varied to adjust to the cell growth rate in order to maintain a constant number of cells in the bioreactor.

The loss of biological cells when operating the bioreactor in the continuous chemostat mode typically decreases the productivity and increases the expense of operating the bioreactor. As a result, a number of modifications to reduce loss of cells have been attempted. The modifications include the incorporation of methods to retain cells in the bioreactor. Unfortunately, cell retention is most difficult when cells are grown in batch suspension culture mode. Anchorage-dependent cells or adherent cells, on the other hand, can be grown on solid matrixes, such as micro-carriers. Nonetheless, employing micro-carriers to retain cells often creates more technical problems, such as the necessity for maintaining suspension of the micro-carriers.

In addition, mixing of cells, whether the cells are in suspension or immobilized on micro-carriers, typically subjects the cells to additional mechanical shear stress which may result in cell rupture, since mammalian cells are fragile and very sensitive to shear stress. Solutions for retaining cells and/or micro-carriers in the bioreactor during perfusion, such as in-line cell filters which permit fluid to pass but retain the cells have problems with clogging, especially when high molecular weight proteins are present in the culture media (see U.S. Pat. No. 4,166,768 issued to Tolbert). The in-line cell filters also impede flow in the bioreactors, and thus limit the culture media feed rate that perfuses through the bioreactor.

Another approach is to incorporate an in-line settling device, which allows cells to return to the bioreactor by gravity. Unfortunately, bioreactors that include in-line settling devices also limit the culture media feed rate that perfuses through the bioreactor. In addition, the in-line setting devices may restrict the scale-up of the bioreactor.

Cells may also be immobilized by entrapment in solid, non-moving surfaces such as plastic blocks, ceramic matrixes, fibrous material or shavings of plastic or glass wool. Cells may also be immobilized inside micro-capsules, polysaccharide gels, porous beads, or hollow fiber membranes in order to retain them in the bioreactor operating in the continuous perfusion culture mode. Nonetheless, cell immobilization or encapsulation also creates an additional barrier for the diffusion of oxygen to the cells.

Another technical problem with bioreactors operating under the continuous perfusion culture mode is that the high turn over of culture media or the high culture media flow rate through the bioreactor can dilute cell secreted products, which would require additional processing steps. Additional processing steps typically result in an increase in expense since subsequent purification steps would be necessary.

Additionally, cells often secrete protein growth factors that support continued cell growth, differentiation, function and viability. Protein growth factors may be exogenously added to the cell culture in order to support cell growth, as is, or as part of a serum. Unfortunately, bioreactors operating under continuous perfusion culture modes typically dilute the protein growth factors in the culture media, and therefore, add to the expense of operating bioreactors since the protein growth factors require replacement.

Therefore, many prior art bioreactors are limited by problems of nutrient exhaustion, growth factor deprivation and metabolic waste product accumulation. Furthermore, these problems generally increase as the size of the bioreactor increases.

Control of Cell Micro-environment

Mammalian cell cultures typically grow to relatively low densities of about 1 million to about 2 million cells per ml when prepared in bioreactors operating under the batch suspension culture mode. Furthermore, some scientists have observed that operating bioreactors under the continuous perfusion culture mode often enables bioreactors to grow the cells to a higher density than is achieved when operating the bioreactor under the batch suspension culture mode. Indeed, bioreactors operating under continuous perfusion culture mode can increase the cell density by about 3 to about 30 fold.

Unfortunately, growing and maintaining the cell culture at the high density creates additional technical problems. As an example, the amount of nutrients consumed and metabolic waste production produced per unit volume increases in proportion to the increase in cell density. The increase in nutrient consumption and metabolic waste product places a greater requirement for efficient feed and waste removal systems. In addition, increased production of metabolic waste products like increased lactic acid per unit volume fuels a greater requirement for a more sophisticated pH control. Typically, pH is controlled by addition of sodium bicarbonate to the bioreactor. In the presence of high levels of lactate, addition of sodium bicarbonate causes an increase in osmolarity that can be detrimental to cell cultures.

In another example, cell cultures maintained at the high density also consume more oxygen per unit volume. Increased oxygen demand increases requirements for maintaining oxygen saturation levels around the cells. However, maintaining adequate oxygen saturation levels in the bioreactor typically requires the use of gas sparging and/or higher agitation rates that increase hydrodynamic shear forces.

In addition, cells also produce significant levels of carbon dioxide at the high density which can induce cell death if carbon dioxide is allowed to accumulate in the bioreactor. Unfortunately, current gas sparging techniques are designed to increase the amount of oxygen available to cell cultures and not the removal of carbon dioxide from the bioreactor.

Therefore, there exists an urgent need to design a bioreactor system that is effective to deliver adequate oxygen and nutrients to mammalian cells. In addition, an urgent need exists to provide a bioreactor system that efficiently removes metabolic waste products from the culture media without dilution of exogenously added protein growth factors or removal of valuable biological cells. Furthermore, an urgent need exists to control the cellular micro-environment within a bioreactor system so that high cell culture densities may be obtained without concomitant destruction of cell culture viability.

### BRIEF SUMMARY OF THE INVENTION

The present invention relates to a bioreactor support system having a cell retention module, an integrating module and a reconditioning media loop. The cell retention module is in communication with a bioreactor which has a cell culturing chamber. The chamber has ingress for oxygenated waste-free cell culturing media and egress for waste-containing, oxygen-depleted media. The integrating module provides to the bioreactor the oxygenated waste-free cell culturing media while accepts the waste-containing, oxygen depleted-media and provides such waste-containing, oxygen-depleted media to the reconditioning media loop. The reconditioning media loop includes a media oxygenator and can also include a dialysis device. The media within the reconditioning loop is circulated through the oxygenator and the dialysis device at a flow rate greater than the flow rate suitable for culturing cells and thereby provides on a continuous basis to the integrating module, and in turn to the bioreactor, oxygenated media with waste removed suitable for culturing cells.

### BRIEF DESCRIPTION OF THE DRAWINGS

FIG. 1 illustrates a bioreactor system in accordance with the present invention.

FIG. 2 illustrates an alternate embodiment of a bioreactor system in accordance with the present invention.

### DETAILED DESCRIPTION

The present invention generally relates to a system and method for growing or maintaining biological cells in vitro. More specifically, the present invention relates to a bioreactor system and method that are effective to grow or maintain biological cells at a high cell density, while providing adequate oxygen and nutrients to, and removing waste products, from the biological cells.

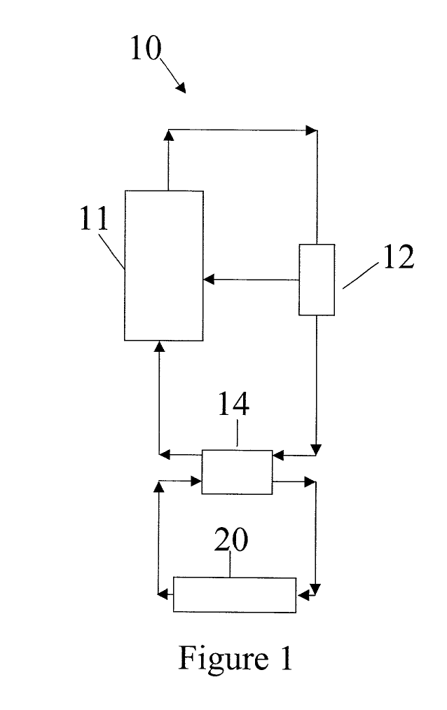

A bioreactor system is generally depicted at 10 in FIG. 1. The bioreactor system includes:

(a) a bioreactor 11 in communication with a cell retention module 12;

(b) an integrating module 14 for collecting cell-free conditioned culture media;

(c) a high speed circulation loop containing an artificial lung device 20 (oxygenator) connected to the integrating module 14;

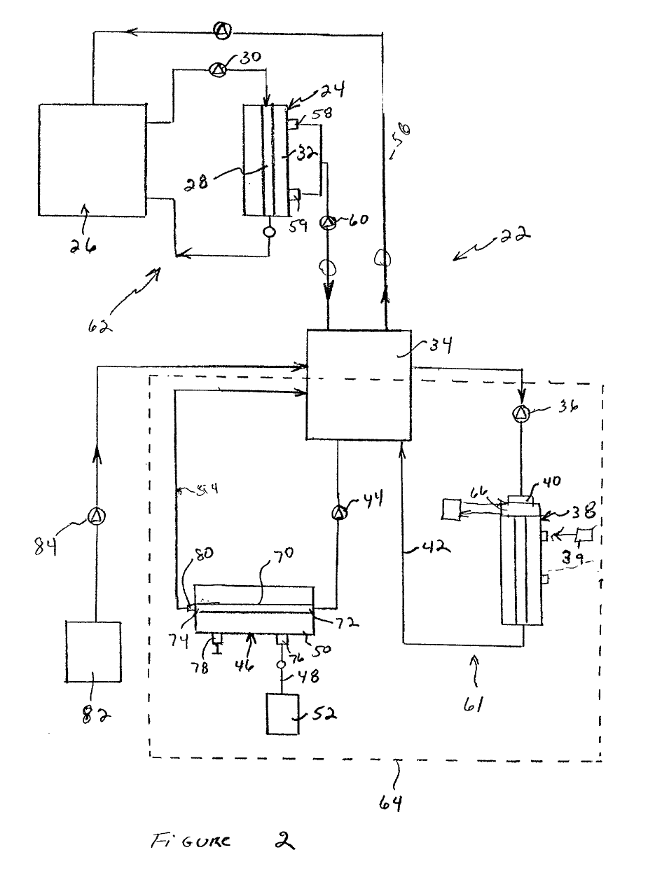

In an alternate embodiment, FIG. 2 illustrates a bioreactor system that includes:

(a) a microporous hollow-fiber cell retention module 24 connected to a bioreactor 26 where the cell-containing culture media is slowly pumped through the lumen of the hollow fibers 28 of the cell retention module 24;

(b) a pump 30 or other motive force that facilitates movement of culture media from the extra-capillary space of the cell retention module 32 to an integrating module 34;

(c) the integrating module 34 which receives the cell-free culture media from the extra-capillary space 32 of the cell retention module 24;

(d) a high speed pump 36 connected to an artificial lung 38 (oxygenation module);

(e) a heat exchange device 40;

(f) a return line 42 from the artificial lung 38 to the integrating module 34 that returns oxygen-saturated, pH-adjusted culture media;

(g) a high speed pump 44 connected to a lumen side of an artificial kidney (dialysis module) 46;

(h) a waste line 48 connected to a shell side 50 of the artificial kidney 46 for removal of metabolic waste products 52;

(i) a return line 54 from the artificial kidney 46 to the integrating module 34 that returns high molecular weight proteins and metabolic waste-depleted media;

(j) a return line 56 from the integrating module 34 to the bioreactor 26 that provides oxygen-saturated, waste-depleted conditioned culture media.

Cell Retention Module

The cell retention module 24 functions to retain and/or separate cells from culture media when using the bioreactor system 10 of the present invention. Preferably, the cell retention module 24 is a membrane device, such as a hollow fiber membrane cartridge that provides a barrier to separate and/or cells from culture media components. The hollow fiber membrane device is selected to have a membrane molecular weight cut-off of less than a size of the cells so that cells will remain on one side of the membrane, while media can freely flow across or through the membrane.

Selection of membranes with a higher porosity typically improve culture media flow and decrease the possibility of clogging of the hollow fiber pores. In addition, it is desirable to select a hollow fiber membrane cartridge with a higher surface area in order to prevent clogging of the hollow fiber membrane. Preferably, the molecular weight cut-off of the hollow fiber membranes is in the ultrafiltration range of 0.2 to 0.65 microns.

Cell-containing culture media that is removed from the bioreactor 26 is delivered to the cell retention module 24, and preferably to the lumen side of the hollow fibers. A wider inner diameter of the hollow fibers is desirable, such as an inner diameter in excess of 250 microns, in order to avoid excessive shear on the cells trafficking through the lumen side of the hollow fibers.

Though descriptions of the present invention are primarily made in terms of the preferred cell retention module 24 that includes a hollow fiber membrane cartridge, it is to be understood that any other cell retention device, such as settling devices, may be substituted in place of the cell retention module 24 in accordance with the present invention while still realizing benefits of the present invention. Likewise, it is to be understood that any combination of any hollow fiber membrane cartridge and any other cell retention module may be used in accordance with the present invention, while still realizing the benefits of the present invention.

The cell retention module 24 may be placed at any position proximate the bioreactor 26, the integrating module 24, the artificial lung 38 or the artificial kidney 46 when practicing the present invention so long as the cell retention module 24 is effective to retain cells. Preferably, the cell retention module 24 is placed above the bioreactor 26 to create a settling effect on the cells traveling through the cell retention module 24. The cells, therefore, travel against gravity when passing through the cell retention module 24 to minimize the percentage of cells which travel through the cell retention module 24, and thus minimize the risk of damage to large numbers of cells trafficking through the cell retention module 24.

Cell-free culture media is removed from the cell retention module 24 through the extra-capillary ports 58 and 59 on the shell side of the cell retention module 24 as best depicted in FIG. 2. Preferably, the cell-free culture media is removed by a pump 60 connected to the extra-capillary ports 58, 59. The pump 60 may be operated at pre-determined on-off intervals or continuously. As an example, minimum cell damage is attained when the pump 60 is on for 1 minute and off for the amount of time it takes for media to travel from one end of the cell retention module to the other (not shown). The time maybe varied by adjusting the rate of the pump 30 which delivers cell-containing culture media from the bioreactor 26 to the cell retention module 24 and/or by selecting a shorter hollow fiber membrane cartridge. In addition, the off-cycle permits flushing off cells from the membranes that may have been retained on the fiber walls due to the hydrostatic force caused by the removal of culture media radially across the hollow fiber membrane.

Integrating Module

The integrating module 34 generally serves as an interface between the slow speed perfusion loop 62 that delivers culture media to the bioreactor 26 and the high speed loop 64 which delivers culture media to the oxygenation module 38 and the dialysis module 46. As an example, the integrating module 34 may be a spinner flask with a cover designed to accommodate all entry and exit ports for a fluid path into the bioreactor 26, or a plastic, disposable vessel. Preferably, a volume of the integrating module 34 ranges between about 25 to about 50% of the volume of the bioreactor 26.

High Speed Circulation Loop Containing an Artificial Lung

Culture media is oxygenated in a high flow rate circuit 61 from the integrating module 34 to the oxygenation module 38 and then back to the integrating module 34. The oxygenation module 38 or artificial lung 38 maybe a gas-permeable hollow fiber cartridge. As an example, an artificial lung device such as the Capiox SX 10 module manufactured by Terumo may be used to provide oxygen, remove carbon dioxide and control pH. For efficient oxygen transfer to the culture media, the rate of culture media flow through the oxygenation module 38 may range from between about 1 liter per minute (L/min) to about 4 L/min. At about 1 L/min approximately 80 milliliters per minutes (ml/min) of oxygen is transferred into the media. At about 4 L/min, the transfer rate of oxygen increases to over 245 ml/min. In addition, the oxygenation module 38 contains a heat exchange component 40 that enables warming of the fluid path without the need for placing the oxygenation module 38 in an incubator. A circulating water bath 66 is connected to the heat exchanger 40 in order to maintain physiological temperature for the cell cultures. If the volume of the bioreactor 26 is too large and/or the perfusion rate too low, it may be necessary to provide an additional heat source to the bioreactor 26 in order to effectively control the temperature.

A controlled gas mixture is introduced into the oxygenation module 38 through amass control device 39. Generally, a mixture of between about 0% and about 10% carbon dioxide in air is adequate for pH control and oxygen saturation. In addition, the gas mixture may be passed through the mass control device 39 at a rate of about 100 ml/min, for example.

The high culture media flow rates required for adequate oxygen transfer rates have prevented the use of this type of oxygenation method for bioreactor processes in the past. As an example, cell culturing media rates of about 31 ml/minute is required for the bioreactor versus about the 1000 ml/min to about 4000 ml/min that is required to attain good oxygen transfer into the culturing media. Such high flow rates of about 1000 ml/min to about 4000 ml/minute create too much shear and destroy fragile mammalian cells. Therefore, the present invention separates the high speed flow from the bioreactor perfusion loop 62 and provides a reservoir of readily available high oxygen-containing culture media in the integration module 34. This high oxygen-containing culture media can then be pumped slowly through the bioreactor 26 in loop 62 to provide saturated oxygen without excessive shear of the cells.

High Speed Circulation Loop Containing an Artificial Kidney

In order to remove metabolic waste products from the culture media while retaining exogenous and/or endogenous growth factors, a hollow fiber dialysis cartridge 70 may used as the dialysis module 46. The dialysis cartridge 70 is placed in a high speed loop 64. The dialysis cartridge 70 functions as an artificial kidney. The artificial kidney 46 may be connected to the integration module 34 independently, as illustrated in FIG. 2, or in series with the oxygenation module 38. When the artificial kidney 46 is placed in series with the artificial lung 38, the artificial kidney 46 is preferably placed upstream from the artificial lung 38 in order to increase the back pressure on the artificial kidney 46. The flow through the porous hollow fibers 70 of the artificial kidney 46 creates a pressure drop axially along the length of the fibers. The pressure drop causes fluid motility due to forces known as the Starling Effect.

Referring back to FIG. 2, under normal operating conditions, culture media would cross the membranes of the hollow fibers 70 on the front high pressure end 72 of the dialysis cartridge 46 and return to the lumen at the back low pressure end 74 of the dialysis cartridge 46. In order to achieve flow, and thus waste product removal from the lumen 70 to the extra-capillary ports 76, 78, the low pressure port 78 is occluded and the exit tubing inner diameter is narrowed by restriction 80 in order to create a back pressure motive force.

The molecular weight cut-off of the hollow fibers 70 may be less than 10,000 daltons, and more preferably is between 5,000 daltons and 6,000 daltons. Combining high speed recirculation rates through the lumen of the hollow fibers 70 and the restriction 80 on the exit port of the dialysis cartridge 70 increases the pressure drop across the dialysis cartridge 70, and generates sufficient flow for effective waste product removal.

Metabolic waste products of the cells typically have molecular weights less than 5,000 daltons, while endogenous and exogenous growth factors have molecular weights that generally exceed 10,000 daltons. Thus, the artificial kidney 46 is capable of removing metabolic waste products while retaining exogenous and endogenous growth factors. Media-containing metabolic waste products that is removed from the artificial kidney 46 can be replaced in the fluid circuit by addition to the integrating module 34 from a source 82 using a pump 84. In this manner, the cells in the bioreactor 26 can be fed and cleared of metabolic waste products without dilution of exogenous and endogenous growth factors. At the same time secreted products from the cells will accumulate and concentrate in the media and will not be diluted by the feeding.

Overall System

The bioreactor vessel 26 can be any type of commercially available housing for the culture of mammalian cells, including fermentation vessels, flexible bags and hollow fiber modules. A variable speed pump 30 may comprise a centrifugal pump, positive displacement pump or gear pump. The pump 30 is in fluid communication with the bioreactor 26 and the lumen side of the cell retention module 24. The pump 30 delivers media containing cells, metabolic waste, cell produced products, exogenously added factors to the cell retention module 24, preferably entering from the lumen side of the cell retention module 24. A valve 25 is placed on the lumenal exit side of the cell retention module 24 which is fluidly connected to the bioreactor 26 as a return line 27. When valve 25 is engaged so as to block the fluid flow from the lumen exit of the cell retention module 24, the media from the bioreactor will pass from the lumen of the cell retention module 24 to the extra-capillary space 32. Due to the pore size of the capillaries (hollow fibers) of the cell retention module 24, the media containing metabolic waste products, cell produced products and exogenously added factors will pass through the fibers and enter the integrating module 34. The cells will remain on the lumen side of the hollow fibers of the cell retention module 24.

Furthermore, valve 25 is operated to intermittently open and close. In the open mode, cells are returned to the bioreactor 26. In the closed mode, cell-free media is delivered back to the integrating module 34 for reconditioning.

The integrating module 34 is connected to a high speed circulation loop 64. The pump 36 may be operated to provide circulation of about 2 liters per minute to about 4 liters per minute. Cell free culture media from the integrating module 34 entering the high speed circulation loop 64 is first delivered to the lumen side of an artificial lung device 38. The artificial lung 38 is preferably constructed from a hollow fiber cartridge. Controlled gases are introduced into the extra-capillary side of the artificial lung 38 to replenish the oxygen and adjust the pH of the cell-free media by removal of carbon dioxide. The oxygenated and pH adjusted cell-free culture media is then returned back to the integrating module 34. Waste containing media is drawn from the integrating module 34 by pump 44 and passed through the lumen side of an artificial kidney device 46. The artificial kidney device is of a hollow fiber cartridge construction having a plurality of hollow fibers extending therethrough within a housing that defines an extra-capillary space between the housing and the hollow fibers. The cartridge contains the ports 76 and 78, port 76 being the high pressure extra-capillary port while port 78 is the low pressure extra-capillary port. The low pressure extra-capillary port 78 is blocked. The high pressure extra-capillary port 76 is connected to a waste container 52 by line 48 and valve 77. Opening valve 77 allows waste media to enter the waste container 52. The low molecular weight cut-off of the hollow fibers within the artificial kidney 46 retain all exogenously added factors and cell produced products while allowing metabolic waste products to be removed from the culture media. The metabolic waste removed media is then returned to the integration module 34.

Fresh nutrient culture media may also be delivered to the integrating module 34 from a fresh media container 82 and delivered by pump 84. Nutrient-replenished, oxygen-replenished, pH-adjusted, waste-depleted media is then returned to the bioreactor 26 by pump 31 which is in fluid communication with the integrating module 34 and the bioreactor 26.

The present invention provides an improved method to deliver oxygen to mammalian cells in a bioreactor providing greater oxygen transfer with less shear. In addition, the present invention provides an improved, universal method of operating bioreactors of any type in a continuous perfusion culture mode while retaining cells in the bioreactor, providing efficient removal of metabolic waste products, and providing a means to supply nutrients without dilution of exogenously added serum or proteins or endogenously produced protein factors.

The present invention further provides an improved control of the cell micro-environment in order to enable efficient high density cell culture in a bioreactor. Specifically, the bioreactor system provides efficiently oxygenated media to a bioreactor, maintains precise control of media pH and osmolarity and efficiently removes carbon dioxide from the media. This invention also provides a method to scale-up bioreactors without the prior limitations of oxygen availability, nutrient availability, removal of metabolic waste and control of cellular microenvironment.

The following example is intended for illustrative purposes only and does not limit the present invention in anyway.

### EXAMPLES

A 1.5 Liter Celligen (New Brunswick Scientific, Edison, N.J.) bioreactor system equipped with two marine impellers and a six-blade Rushton turbine positioned at the gas/liquid interface to increase gas transfer capability (the agitation at the surface increases the liquid/gas surface area) was set at an agitation speed of about 100 revolutions per minutes. Perfusion was accomplished by recirculating the culture media through a external hollow-fiber module (CellFlo, Spectrum, Laguna Hills, Calif.). The total working volume of the system was 1.7 Liters, including the volume of the recirculation loop. The dissolved oxygen was set to be maintained at 40%.

1×10⁷ HL-60 leukemia cells were inoculated in the system in X-Vivo 15 serum-free media (BioWhittiker) supplemented with 2 mM glutamine, provides an initial cell density of approximately 0.5×10⁶ cells/ml. Samples from the bioreactor were taken twice daily and analyzed for total cell number, viability, glucose, lactate and ammonia levels. The culture was perfused at 0.5 volume per volume per day with fresh media.

After 5 days in culture the cell density reached a peak of 5×10⁶ cells/ml. By day 7, the viable cell number had decreased over 60%. By introducing sparging on day 9 at 0.008 volume per volume per minute, the viable cell density increased to 7×10⁶ cells per ml on day 14. However, the ratio of alive:dead cells in the reactor steadily decreased from 1:2 on day 14 to 1:4 on day 16.

When the same experiment was conducted with the oxygen enrichment system of the present invention, the culture density reached 9×10⁶ cells/ml on day 7. The alive:dead ratio was 10:1 on day 7, and no sparging was required. By day 14, the culture had reached a steady state at 3×10⁷ cells/ml and the alive:dead cell ratio was 9:1. These results demonstrate that the present invention is capable of enhancing the oxygen availability to cells in perfusion culture. The enhanced oxygen delivery for a given rate of perfusion can maintain cells at higher density without the need for sparging, which increases shear and viability of the cells in the culture.

Although the present invention has been described with reference to preferred embodiments, workers skilled in the art will recognize that changes may be made in form and detail without departing from the spirit and scope of the invention.

1. A bioreactor support system for supporting a bioreactor, the bioreactor support system comprising:
a cell retention module in communication with the bioreactor having a cell culturing chamber, the chamber having ingress for oxygenated waste-free cell culturing media and egress for waste-containing, oxygen-depleted media;
an integrating module in fluid communication with the chamber providing oxygenated waste-free culturing media to the chamber and accepting oxygen waste-containing depleted media;
a reconditioning media loop including a media oxygenator and a dialysis device, the reconditioning loop providing on a continuous basis oxygenated culturing media to the integrating module and accepting for reconditioning the waste-containing, oxygen-depleted media to be circulated through the oxygenator; and
wherein the reconditioning media loop is at a flow rate greater than a flow rate of culturing media in the bioreactor.

2. The bioreactor support system of claim 1 wherein the dialysis device is constructed to retain cell growth factors while removing waste from the culturing media.

3. The bioreactor support system of claim 1 wherein the retention module has at least one membrane having a molecular weight cut-off that is less than the size of the cells being cultured to thereby retain the cells.

4. The bioreactor support system of claim 1 wherein the flow rate of the reconditioning loop is at least about two times greater than the flow rate of the culturing media in the bioreactor.

5. The bioreactor support system of claim 1 wherein the reconditioning media loop includes an oxygenating loop portion and a waste removal loop portion.

6. The bioreactor support system of claim 5 wherein the oxygenating loop portion and the waste removal loop portions are operated independently, each portion having a pump at a flow rate that is at least about two times greater than the flow rate of the culturing media in the bioreactor and the cell retention module.

7. The bioreactor support system of claim 1 wherein the cell retention module includes a pump and at least one control for operating the pump in an on/off status.

8. The bioreactor support system of claim 1 wherein the oxygenator controls pH by the addition of carbon dioxide.

9. A bioreactor support system for supporting a bioreactor, the bioreactor support system comprising:
a cell retention module in communication with the bioreactor having a cell culturing chamber, the chamber having ingress for oxygenated waste-free cell culturing media and egress for waste-containing, oxygen-depleted media;
an integrating module in fluid communication with the bioreactor providing oxygenated waste-free media to the bioreactor and accepting waste-containing, oxygen depleted media;
a reconditioning media loop including a media oxygenator and a dialysis device, the reconditioning loop being in fluid communication with the integrating module; and
the media being circulated through the oxygenator and the dialysis device at a flow rate greater than the flow rate suitable for culturing cells.

10. The bioreactor support system of claim 9 wherein the dialysis device is constructed to retain cell growth factors while removing waste from the media.

11. The bioreactor support system of claim 9 wherein the retention module has at least one membrane having a molecular weight cutoff that is less than the size of the cells being cultured to thereby retain the cells.

12. The bioreactor support system of claim 9 wherein the flow rate of the reconditioning media loop is at least two times greater than the flow rate of the media in the bioreactor.

13. The bioreactor support system of claim 9 wherein the reconditioning media loop includes an oxygenating loop portion and a waste removal loop portion.

14. The bioreactor support system of claim 13 wherein the oxygenating loop portion and the waste removal loop portions are operated independently, each portion having a pump for moving the media at least two times greater than the flow rate of the media in the bioreactor and the cell retention module.

15. The bioreactor support system of claim 13 wherein the cell retention module includes a pump and at least one control for operating the pump in an on/off status.

16. The bioreactor support system of claim 13 wherein the oxygenator control pH by the addition of carbon dioxide

17. A bioreactor support system for supporting a bioreactor comprising:
a cell retention module in fluid communication with a bioreactor having a wall enclosed cell culturing chamber, the chamber having ingress for oxygenated waste-free cell culturing media and egress for waste-containing, oxygen-depleted media;
an integrating module in fluid communication with the bioreactor providing oxygenated waste-free media to the cell retention module and accepting waste-containing, oxygen depleted media;
an oxygenator in fluid communication with the integrating module for oxygenating media taken from the integrating module and returning oxygenated media to the integrating module;
a waste removal device in fluid communication with the integrating module, the waste removal device taking media from the integrating module and removing waste therefrom and returning with substantially waste-free media to the integrating module; and
wherein the media being oxygenated and having waste removed is flowing to and from the oxygenator and the waste removal device at a flow rate that would be detrimental to the culturing of cells while media moving through the bioreactor is moving at a flow rate that is not detrimental to the culturing of cells.

18. The bioreactor support system of claim 17 wherein the cell retention module is a hollow fiber cartridge containing a plurality of hollow fibers surrounded by a shell defining an extracapillary space and where the ingress and egress are to the extracapillary space.

19. The bioreactor support system of claim 17 wherein the oxygenator is a hollow fiber cartridge having a plurality of hollow fibers therein surrounded by a shell defining an extracapillary space between the shell and the fibers and wherein the media to be oxygenated flows through lumens of the hollow fibers.

20. The bioreactor support system of claim 17 wherein the cell retention module is a hollow fiber cartridge having a plurality of hollow fibers therein, the hollow fibers having lumens, and a shell surrounding the hollow fibers and defining an extracapillary space between the wall and the hollow fibers, and wherein cells being cultured traverse through the lumens of the hollow fibers and are prevented from entering the extracapillary space by membrane walls of the hollow fibers.

21. The bioreactor support system of claim 17 wherein the waste removal device is a hollow fiber cartridge having a plurality of hollow fibers surrounded by a wall, defining an extracapillary space between the wall and the hollow fibers and wherein the media from which waste is to be removed flows through lumens of the hollow fibers, with the hollow fibers having membrane walls permitting waste to flow therethrough while retaining cell growth factors within the media flowing through the lumens.

22. The bioreactor support system of claim 17 wherein the flow rate of media being supplied to the oxygenator and to the waste removal device is at least twice the flow rate of the media within the lumen of the cell retention module.

23. The bioreactor support system of claim 17 wherein the oxygenator and the waste removal device are each positioned in separate flow loops, each loop having its own pump.

24. The bioreactor support system of claim 17 wherein the oxygenator and the waste removal device are positioned in series in the same flow loop.

25. The bioreactor support system of claim 24 wherein the oxygenator is disposed downstream of the waste removal device.

26. A bioreactor support system for supporting a bioreactor comprising:
a first media flow loop at a first media flow rate in which media is used to culture mammalian cells;
a second media flow loop in which media flows at a second flow rate and in which media is reconditioned so as to be suitable for culturing mammalian cells;
an integration module fluidly connected to the first media flow loop for providing media suitable for culturing mammalian cells and receiving media therefrom for reconditioning and fluidly connected to the second media flow loop for providing media to be reconditioned and accepting reconditioned media; and
wherein the second flow loop has a flow rate that is higher than the flow rate in the first flow loop and which is deleterious to the culturing of mammalian cells.

27. The bioreactor support system of claim 26 wherein the second flow rate is at least twice in magnitude as the first flow rate.

28. The bioreactor support system of claim 26 wherein the first media flow loop retains cells being cultured therein while permitting oxygen-depleted, waste-containing media to flow to the integrating module.

29. The bioreactor support system of claim 26 wherein the second media flow loop reconditions oxygen-depleted, waste-containing media so as to be suitable for culturing mammalian cells while retaining within the media cell growth factors.

30. A bioreactor system, the bioreactor system comprising:
a bioreactor having cell culturing chamber, the chamber having ingress for oxygenated cell culturing media and egress for oxygen-depleted media;
an integrating module in fluid communication with the chamber providing oxygenated cell culturing media to the chamber and accepting oxygen depleted media;
a reconditioning media loop including a media oxygenator, wherein the reconditioning loop provides oxygenated cell culturing media to the integrating module, and wherein the reconditioning loop oxygenates the oxygen-depleted media; and
wherein the reconditioning media loop is at a flow rate greater than a flow rate of culturing media in the bioreactor.

31. The bioreactor system of claim 30 wherein the bioreactor further includes a cell retention module that is effective to retain cells.

32. The bioreactor system of claim 30 wherein the reconditioning media loop further includes a dialysis device that is effective to retain cell growth factors while removing waste from the culturing media.

33. The bioreactor system of claim 31 wherein the cell retention module includes at least one membrane having a molecular weight cut-off that is less than the size of the cells being cultured to thereby retain the cells.

34. The bioreactor system of claim 30 wherein the flow rate of the reconditioning media loop is at least about two times greater than the flow rate of the culturing media in the bioreactor.

36. The bioreactor system of claim 30 wherein the cell retention module includes a pump and at least one control for operating the pump in an on/off status.

37. The bioreactor system of claim 30 wherein the oxygenator controls pH by the addition of carbon dioxide.
